(19) United States
(12) Patent Application Publication
(10) Pub. No.: US 2013/0231312 A1
(43) Pub. Date: Sep. 5, 2013

(54) TREATMENT OF TYPE II DIABETES AND DIABETES-ASSOCIATED DISEASES WITH SAFE CHEMICAL MITOCHONDRIAL UNCOUPLERS

(71) Applicant: Shengkan Jin, Piscataway, NJ (US)

(72) Inventor: Shengkan Jin, Piscataway, NJ (US)

(21) Appl. No.: 13/885,638

(22) PCT Filed: Nov. 16, 2011
(86) PCT No.: PCT/US11/61028

§ 371 Date: May 15, 2013

Related U.S. Application Data
(60) Provisional application No. 61/414,030, filed on Nov. 16, 2010.

(51) Int. Cl. A61K 31/609; A61K 45/06

(52) U.S. Cl. CPC A61K 31/609; A61K 45/06
USPC 514/166

(57) ABSTRACT

This application discloses methods for treating, preventing and/or alleviating the symptoms of type II diabetes and diabetes-related disorders or complications. The invention provides a novel approach to treating and managing disorders and symptoms related to elevated plasma glucose concentrations and insulin resistance, characterized by few side effects and low toxicity. In particular, the invention provides 2-hydroxy-benzoic anilide compounds and derivatives, and compositions thereof, which can control blood-glucose and increase insulin sensitivity by reducing plasma glucose concentration and cellular energy efficiency.

### CROSS-REFERENCE TO RELATED APPLICATIONS

This application claims priority under 35 U.S.C. §119(e) to U.S. Provisional Patent Application Ser. No. 61/414,030, filed on Nov. 16, 2010, which is hereby incorporated by reference in its entirety.

### STATEMENT REGARDING FEDERALLY SPONSORED RESEARCH

The invention described herein was supported in whole or in part by grants from the National Institutes of Health (Grant Nos. 1R01CA116088 and 1R01AG030081). The U.S. Government has certain rights in this invention.

### FIELD OF THE INVENTION

The present invention relates to compounds, compositions and new methods for treating and/or preventing type II diabetes and related disorders and complications through uncoupling mitochondria.

### BACKGROUND OF THE INVENTION

Type II diabetes is an adult-onset metabolic disease characterized by elevated plasma glucose concentration and insulin resistance of peripheral tissues. Type II diabetes inflicts about 20 million people in the US alone. If the hyperglycemic conditions in the type II diabetic patients are not intensively controlled pharmacologically, severe and sometimes fatal complications, such as cardiovascular diseases, heart attack, kidney failure, gastrointestinal diseases, gangrene, and blindness, can rapidly develop in patients. Obesity increases the risk of developing type II diabetes; however, obesity is not sufficient to cause type II diabetes, nor is obesity required for the development of type II diabetes. A majority of obese individuals do not develop diabetes; and type II diabetic patients are not always obese. Recent studies in type II diabetes research showed that it is not obesity per se that causes type II diabetes. Instead it is the abnormal accumulation of lipid in liver and skeletal muscles that plays a causal role in the development of insulin resistance and hence type II diabetes. (Samuel V. T., et al., Lancet, 2010, 375:2267-77).

Unfortunately, currently there are no cures for type II diabetes. Type II diabetic patients rely on pharmacotherapy for controlling the hyperglycemic symptom for the rest of their lives. Currently a number of drugs are available targeting various processes involved in glycemic control, including metformin (inhibiting hepatic gluconeogenesis), sulfonylureas (increasing insulin secretion), thiazolidinediones (improving adipose lipid metabolism), glucosidase inhibitors (reducing glucose absorption), GLP-1 analogs, amylin analogs, DPP-4 inhibitors (all increase satiety and reduce glucagon), and insulin supplementation. These drugs are used as monotherapies or in combination. However, resistance or tolerance to these treatments will eventually develop in patients. Therefore, development of new anti-diabetic drugs with novel mechanisms of action, which can either be used as monotherapies and/or used in combination with existing regimens to delay the progression of the disease, is a priority of research in improving diabetes therapy.

### SUMMARY OF THE INVENTION

The present invention is designed to improve diabetes therapy by providing compositions and methods for treatment and prevention of type II diabetes and obesity. It provides new clinical means of blood glucose control, by using the compound, 2′,5-dichloro-4′ -nitro salicylic anilide and related compounds, which have mitochondrial uncoupling activities, to effectively reduce plasma glucose concentrations, increase insulin sensitivity, and reduce cellular energy efficiency, without the side effects and shortcomings of current methods of treatment.

The family of 2-hydroxy-benzoic anilide compounds, some of which were previously used as anthelmintics, have been shown to be efficacious in treating and preventing type II diabetes when administered to mice. Acute administration of these compounds effectively reduces plasma glucose concentrations. Long-term oral administration increases insulin sensitivity, reduces fasting glucose and insulin levels. These outcomes result from mitochondrial uncoupling and disruption of the mitochondrial energy cycle. When analyzed on isolated mitochondria or in cell culture at concentrations comparable to plasma concentrations in vivo, these compounds uncouple mitochondria and stimulate oxidation of mitochondrial fuels without ATP production. The chemical modification that abolishes mitochondrial uncoupling activity of these compounds also abolishes the anti-diabetic effect. The safety of some members in this compound family in rodents and humans is well-established.

Thus, in one aspect the present invention provides use of a family of compounds in the treatment and prevention of type II diabetes and related disorders and complications, derived from the classes of compounds including, but not limited to, 2-hydroxy-benzoic anilide compounds, benzimidazoles, N-phenylanthranilates, phenylhydrazones, salicylic acids, acyldithiocarbazates, cumarins, and aromatic amines that have mitochondrial uncoupling activities.

In another aspect, the present invention provides a method of treatment of type II diabetes and its symptoms, using the family of 2-hydroxy-benzoic anilide compounds or other mitochondrial uncouplers. In particular, the 2-hydroxy-benzoic anilide compounds suitable for use in the present invention include compounds of formula (I):

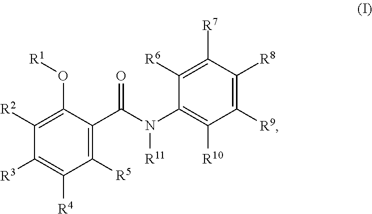

or pharmaceutically acceptable salts, solvates, or prodrugs thereof, wherein

R¹ and R¹¹ are each independently hydrogen (H) or a protecting group that can be hydrolyzed in vivo to become hydrogen;

R² through R¹⁰ are each independently selected from hydrogen, halogen, —CN, —NO₂, —NR^{a}R^{b}, C₁-C₆ alkyl, C₁-C₆ alkenyl, C₁-C₆ alkynyl, C₁ -C₆ haloalkyl, —OR²⁰, —C(O)R²¹, and —OC(O)R²², wherein R²⁰, R²¹ and R²² are each independently hydrogen, C₁-C₆ alkyl or C₁-C₆ alkenyl, and wherein each said alkyl, alkenyl, alkynyl, or haloalkyl is optionally substituted with one, two, or three substituents independently selected from halogen, hydroxyl, C₁-C₄ alkoxy, —CN, —NH₂, —NO₂, and oxo (═O); and

R^{a} and R^{b} are each independently hydrogen or C₁-C₆ alkyl;

wherein at least one of R² through R⁵ is not hydrogen, and at least one of R⁶ through R¹⁰ is not hydrogen.

In another aspect, the present invention provides a method of preventing metabolic and metabolism-related diseases or disorders, including, but not limited to, pre-type II diabetes, type II diabetes, obesity and obesity-related disorders and complications.

In another aspect, the present invention provides a method of using these compounds to treat obesity, its symptoms and related conditions, including, but not limited to type II diabetes.

In another aspect, the present invention provides a new approach for long-term chronic disease management by reducing plasma glucose.

In another aspect the present invention provides compounds of formula (I):

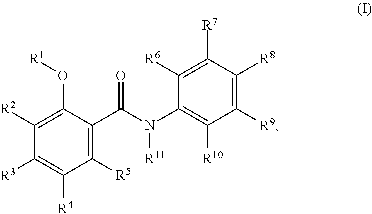

or pharmaceutically acceptable salts, solvates, or prodrugs thereof, wherein

R¹ and R¹¹ are each independently hydrogen (H) or a protecting group that can be hydrolyzed in vivo to become hydrogen;

R² through R⁵ are independent, at least one of which is selected from the group consisting of —OH, halogen, —CN, —NO₂, —CH(CH₃)₂, —C(CH₃)₃, and trihalo-methyl; and the rest of which are selected from —H, C₁₋₆alkyl, C₁₋₆alkenyl, C₁₋₆alkynyl, C₁₋₆alkoxy, C₁₋₆haloalkyl, hydroxyC₁₋₆alkyl, heteroaryl, and phenyl, wherein said heteroaryl or phenyl is optionally substituted with one to five substituents independently selected from Cl, Br, F, CF₃, and methoxy;

R⁶ through R¹⁰ are independent, at least one of which is selected from the group consisting of —OH, halogen, —CN, —NO₂, —CH(CH₃)₂, —C(CH₃)₃, and trihalo-methyl; and the rest of which are selected from —H, C₁₋₆alkyl, C₁₋₆alkenyl, C₁₋₆alkynyl, C₁₋₆alkoxy, C₁₋₆haloalkyl, hydroxyC₁₋₆alkyl, heteroaryl, and phenyl, wherein said heteroaryl or phenyl is optionally substituted with one to five substituents independently selected from Cl, Br, F, CF₃, and methoxy.

In another aspect the present invention provides compositions containing any of the compounds described above.

In another aspect the present invention provides use of the above-defined compounds in treatment or prevention of diabetes, in particular, type-II diabetes, and diabetes-related diseases or complications.

In another aspect the present invention provides use of the above-defined compounds or compositions for long-term management of diabetes and related diseases or complications.

Although the present invention is not limited to any theory of operation, it is believed that type II diabetes is caused by insulin resistance in peripheral tissues and is characterized by hyperglycemia, and reducing blood glucose is the most important therapeutic goal of treating type II diabetes. Mitochondria are critical organelles for glucose and lipid metabolism.

In another particular aspect the present invention uses 5-chloro-salicyl-(2-chloro-4-nitro) anilide 2-aminoethanol salt (CSAA) as mitochondrial uncoupling agent for treatment of diabetic conditions. Intraperitoneal (I.P.) injection of CSAA into db/db diabetic mice or high-fat diet induced pre-diabetic mice leads to effective reduction of plasma glucose levels. This is associated with increased AMPK (5′ adenosine monophosphate-activated protein kinase) activity and increased glucose uptake in liver, skeletal muscles, and other tissues.

In another aspect the present invention provides a method of long-term disease management. Chronic oral treatment by adding CSAA into diet dramatically reduces fasting blood glucose levels in db/db diabetic mice. Similarly, chronic feeding the high-fat diet induced pre-diabetic mice with CSAA greatly reduces fasting blood glucose and insulin levels, and increases insulin sensitivity. The concentrations at which CSAA uncouples mitochondria in cultured cells are within the range of documented plasma CSAA levels after oral administration. Importantly, changing the 2-OH group, which is essential for mitochondrial uncoupling activity, to 2-O—SO₂H, totally abolishes CSAA's efficacy in reducing plasma glucose concentrations. The chronic effect of CSAA on increasing insulin sensitivity is attributable to diminished lipid loads in liver or muscles.

### BRIEF DESCRIPTION OF THE DRAWINGS

FIG. 1 illustrates acute treatment with 5-chloro-salicyl-(2-chloro-4-nitro) anilide 2-aminoethanol salt (CSAA) effectively reducing the blood glucose levels in the diabetic db/db mice and in the high-fat diet induced pre-diabetic mice. (A). Structure of CSAA. Blood glucose concentrations in (B) db/db mice or in (C) C57/B16 mice upon CSAA treatment. The db/db mice at the age of 6 weeks, or the C57/B16 wild type mice fed with high-fat diet (60% fat calorie) for 10 weeks starting from age of 5 weeks, were treated with the CSAA at dosage of 100 microgram/mouse through I.P. route. The mice were starved for 5 hours prior to CSAA injection. The blood glucose concentrations were measured at indicated time points after injection and normalized against the concentration at time 0 (untreated), which was set at 100%. UT, untreated; CSAA, CSAA treated; *, P<0.05; **, P<0.01; ***, P<0.001; n=6 pairs in each set of experiment.

FIG. 2 illustrates acute treatment with CSAA salt activating AMPK and increasing glucose uptake. (A). Levels of the phosphorylated AMPK in liver and muscle tissues upon CSAA treatment. C57/B16 mice were either treated with saline or saline containing CSAA I.P. at the dosage of 100 microgram/mouse. 0, 2, or 4 hours later, mice were sacrificed and liver lysates were analyzed by immunoblotting to detect the levels of phosphorylated AMPK. The levels of the un-phosphorylated AMPK and RAN were also measured as controls. (B). Rates of glucose uptake in various tissues after CSAA treatment. C57/B16 mice were starved for 3 hours, followed by treatment with saline or saline containing CSAA I.P. at the dosage of 100 microgram/mouse. 1.5 hours later, ³H-2-deoxyglucose (0.5 microcurrie/gram body weight) was injected I.P. 30 minutes later, mice were treated with anesthesias and perfused with PBS. Mice were then sacrificed and tissue was extracted for measurement of ³H-2-deoxyglucose accumulation (normalized with tissue mass). UT, untreated; CSAA, CSAA treated; *, P<0.05; **, P<0.01. The data are representative results from two independent experiments.

FIG. 3 illustrates two-week oral CSAA treatment reducing fasting blood glucose concentrations in db/db mice to almost normal levels. (A) fasting glucose concentrations and (B) food uptake in db/db mice. The db/db mice at age of 6 weeks were fed with either normal AIN-93M food or with AIN-93M food containing 1500 ppm of CSAA salt for two weeks. The fasting blood glucose levels and food uptake of the mice were measured (normalized against grams of body weight). UT, Untreated (n=6); CSAA, CSAA treated (n=3), ***P<0.001.

FIG. 4 illustrates chronic oral CSAA treatment reducing blood glucose and insulin levels in high-fat diet induced pre-diabetic mice. Normal C57/B16 mice were fed with high-fat diet (60% fat calorie) or high-fat diet containing 1500 ppm of CSAA for 8 weeks starting at age of 5 weeks. (A) fasting blood glucose concentrations, (B) insulin concentrations, and (C) daily food intake (between weeks 7 to 8 during high-fat diet feeding, normalized against grams of body weight) were measured. UT: untreated; CSAA, CSAA treated; n=8 pairs, ***P<0.001.

FIG. 5 illustrates chronic oral CSAA treatment increasing insulin sensitivity. Normal C57/B16 mice were fed with high-fat diet (60% fat calorie) or high-fat diet containing 1500 ppm of CSAA for 9 weeks starting at age of 5 weeks. Insulin sensitivity was measured by (A) glucose tolerance assay and (B) insulin tolerance assay. UT, untreated; CSAA, CSAA treated; *, P<0.05; **, P<0.01; ***, P<0.001; n=7 pairs.

FIG. 6 illustrates chronic oral CSAA treatment increasing AMPK activity and reduces liver lipid loads. (A). Phosphorylated AMPK levels in liver tissues of mice before and after oral CSAA treatment. The levels of the un-phosphorylated AMPK and RAN were also measured as controls. (B). Representative pictures of H & E stained liver tissues from mice either fed with high-fat diet alone (UT) or fed with high-fat diet containing 1500 ppm CSAA (CSAA) for 10 weeks. The white areas in hepatocytes are cells with high lipid content.

FIGS. 7A and 7B depict the effect of chronic I.P. injections of CSAA to reduce body weight gain when induced by a high-fat diet, without reducing food uptake. 24 normal mice at the age of 8 months were fed with high-fat diet. Half of them (12 mice) were injected daily with CSAA via I.P. route at the dosage of 100 μg/mouse (in 500 μl PBS). The other 12 mice were injected with vehicle only (PBS). The food intake (A) and body weight (B) were measured. *P<0.05, n=12 pairs.

FIG. 8 illustrates mitochondrial uncoupling activities of CSAA, shown with isolated mammalian mitochondria (A) and in cultured mammalian cells (B) and (C). (A). Oxygen consumption chart of mitochondria isolated from mouse liver. Mitochondria, mitochondrial oxidative phosphorylation substrates, indicated inhibitors, and CSAA were added into the respiration chamber in the indicated order. Oxygen consumption was measured with an Oxygraph System. (B) and (C) CSAA reduces mitochondrial membrane potential in mammalian cells. NIH-3T3 cells (˜90% confluence) were treated with CSAA (B) at indicated final concentrations for 2 hours or (C) for indicated period of time at the concentration of 2 μM. The cells were then stained with TMRE (100 nM) for 15 min to detect mitochondria membrane potential. After washed twice with PBS, cells were analyzed under microscope.

FIG. 9 illustrates CSAA increases cellular oxygen consumption in the presence of oligomycin and does not increases cellular ATP levels. (A) Cellular oxygen consumptions were measured continuously for 120 minutes in cells upon treatment with DMSO (control), CSAA (1 μM), oligomycin (5 μg/ml), CSAA and oligomycin. CSAA dramatically increases cellular oxygen consumption even in the presence of oligomycin, indicating its mitochondrial uncoupling activity. (B) ATP concentrations were measured in cells treated with CSAA (1 μM) for indicated period of time. A total 20,000 cells under each condition were seeded and analyzed.

FIG. 10 illustrates the diminished hypoglycemic effect of CSAA after converting 2-OH to 2-O—SO₂H. (A). structure of the sulfite derivative of 5-chloro-salicyl-(2-chloro-4-nitro) anilide. (B). Effect of the sulfite derivative on blood glucose. Pre-diabetic mice were starved for 5 hours followed by I.P. injection of saline or saline containing the CSAA sulfite derivative (100 microgram/mouse). The blood glucose concentrations were measured at indicated time points, and normalized against the concentrations before injection, which was set as 100%. UT, untreated; CSAA, CSAA treated; n=6 pairs.

### DETAILED DESCRIPTION OF THE INVENTION

The present invention relates to novel methods for treating, preventing, and alleviating the symptoms of, type II diabetes and diabetes-related disorders and complications. The family of 2-hydroxy-benzoic anilide compounds and derivatives can be administered as a means of blood-glucose and body-weight control by reducing plasma glucose concentration and cellular energy efficiency.

In one aspect the present invention provides a method of treating or preventing a metabolic disease or disorder in a subject, comprising administering to the subject a therapeutically effective amount of a mitochondrial uncoupling agent.

In one embodiment of this aspect, the mitochondrial uncoupling agent is selected from the group consisting of 2-hydroxy-benzoic anilide compounds, benzimidazoles, N-phenylanthranilates, phenylhydrazones, salicylic acids, acyldithiocarbazates, cumarines, and aromatic amines that have mitochondrial uncoupling activities, or a pharmaceutically acceptable salt, solvate, or prodrug thereof.

In another embodiment of this aspect, the mitochondrial uncoupling agent is a 2-hydroxy-benzoic anilide compound of formula (I):

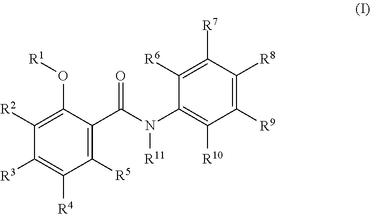

or a pharmaceutically acceptable salt, solvate, or prodrug thereof, wherein:

R¹ and R¹¹ are each independently hydrogen (H) or a protecting group that can be hydrolyzed in vivo to become hydrogen;

R² through R¹° are each independently selected from hydrogen, halogen, —CN, —NO₂,NR^{a}R^{b}, C₁-C₆ alkyl, C₁-C₆ alkenyl, C₁-C₆ alkynyl, C₁-C₆ haloalkyl, —OR²⁰, —C(O)R²¹, and —OC(O)R²², wherein R²⁰, R²¹ and R²² are each independently hydrogen, C₁-C₆ alkyl or C₁-C₆ alkenyl, and wherein each said alkyl, alkenyl, alkynyl, or haloalkyl is optionally substituted with one, two, or three substituents independently selected from halogen, hydroxyl, C₁-C₄ alkoxy, —CN, —NH₂, —NO₂, and oxo (═O); and

R^{a} and R^{b} are each independently hydrogen or C₁-C₆ alkyl;

wherein at least one of R² through R⁵ is not hydrogen, and at least one of R⁶ through R¹⁰ is not hydrogen.

In another embodiment of this aspect, the mitochondrial uncoupling agent is a 2-hydroxy-benzoic anilide compound of formula (I), wherein R¹ and R¹¹ are each independently hydrogen, —C(O)R¹² or —P(P)(OR¹³)R¹⁴, wherein:

R¹² is hydrogen, —OR¹⁵, —NR^{a}R^{b}, C₁-C₂₀ alkyl, C₂-C₂₀ alkenyl, C₆-C₁₀ aryl, or 5- to 10-membered heteroaryl;

R¹⁴ is —OR¹⁵, —NR^{a}R^{b}, C₁-C₂₀ alkyl, C₂-C₂₀ alkenyl, C₆-C₁₀ aryl, or 5- to 10-membered heteroaryl;

R¹³ and R¹⁵ at each occurrence are independently hydrogen, C₁-C₆ alkyl, C₆-C₁₀ aryl, or benzyl; and

R^{a} and R^{b} are each independently hydrogen or C₁-C₆ alkyl,

wherein any said alkyl or alkenyl is optionally substituted by one, two, or three substituents independently selected from hydroxyl, halo, C₁₋₄ alkoxy, and —CO₂R¹⁶; and

wherein any said aryl, heteroaryl, and phenyl part of benzyl is optionally substituted by one to five substituents independently selected from C₁₋₄ alkyl, hydroxyl, halo, C₁₋₄ alkoxy, and —CO₂R¹⁶; and

R¹⁶ is hydrogen or C₁-C₆ alkyl.

In another embodiment of this aspect, the mitochondrial uncoupling agent is a 2-hydroxy-benzoic anilide compound of formula (I), wherein:

R¹¹ is hydrogen;

R¹ is hydrogen or —C(O)R¹², wherein:

R¹² is hydrogen, —OR¹⁵, —NR^{a}R^{b}, C₁-C₈ alkyl, C₂-C₈ alkenyl, or phenyl;

R¹⁵ is hydrogen or C₁-C₆ alkyl; and

R^{a} and R^{b} are each independently hydrogen or C₁-C₆ alkyl.

In another embodiment of this aspect, the mitochondrial uncoupling agent is a 2-hydroxy-benzoic anilide compound of formula (I), wherein R¹ is hydrogen, R^{a}R^{b}N—C(O)—, or an acyl group selected from the group consisting of:

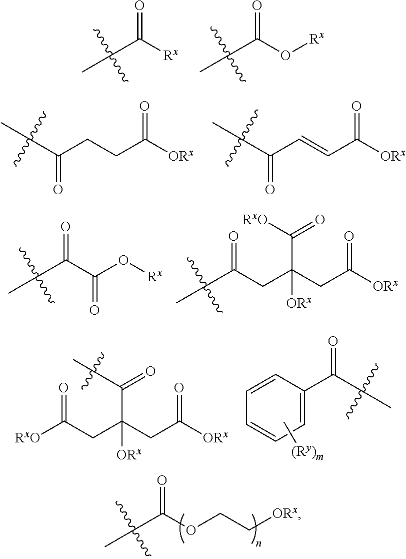

wherein m is 0, 1, 2, 3, 4, or 5;

n is an integer from 1 to 200;

R^{x} at each occurrence is independently hydrogen or C₁-C₈ alkyl; and

R^{y} at each occurrence is independently C₁-C₄ alkyl, halogen, hydroxyl, C₁-C₄ alkoxy, —NO₂, —CN, or—CO₂R^{x}.

In another embodiment of this aspect, the mitochondrial uncoupling agent is a 2-hydroxy-benzoic anilide compound of formula (I), wherein R² through R⁵ are each independently hydrogen, hydroxyl, halo, C₁-C₄ alkyl, C₁-C₄ alkoxy, C₁-C₄ haloalkyl, C₁-C₄ haloalkoxy, and C₁-C₆ acyloxy.

In another embodiment of this aspect, the mitochondrial uncoupling agent is a 2-hydroxy-benzoic anilide compound of formula (I), wherein R¹ is hydrogen or acetyl; R¹¹ is hydrogen; R² through R¹⁰ are each independently selected from the group consisting of hydrogen, hydroxyl, halo, nitro, and methyl.

In another embodiment of this aspect, the mitochondrial uncoupling agent is a 2-hydroxy-benzoic anilide compound of formula (I), wherein R¹ is hydrogen or acetyl; R¹¹ is hydrogen; R² is hydrogen or methyl; R³ is hydrogen; R⁴ is Cl or Br; R⁵ is hydrogen; R⁶ is hydrogen, —Cl, —CH₃, or —NO₂; R⁷ is hydrogen or Cl; R⁸ is —H, —Cl, or —NO₂; R⁹ is H, Cl, or Br; and R¹⁰ is H or Cl.

In another embodiment of this aspect, the mitochondrial uncoupling agent is a 2-hydroxy-benzoic anilide compound of formula (I), wherein the compound is 2′,5-dichloro-4′-nitro salicylic anilide, or a pharmaceutically acceptable salt thereof.

In another embodiment of this aspect, the mitochondrial uncoupling agent is a 2-hydroxy-benzoic anilide compound of formula (I), wherein the compound is 2′,5-dichloro-4′-nitro salicylic anilide 2-aminoethanol salt (CSSA).

In another embodiment of this aspect, the metabolic disease or disorder is related to body-weight control.

In another embodiment of this aspect, the metabolic diseases or disorder is selected from obesity, obesity-related complications, hypertension, cardiovascular disease, nephropathy, and neuropathy.

In another embodiment of this aspect, the metabolic disease or disorder is related to elevated plasma glucose concentrations.

In another embodiment of this aspect, the metabolic disease or disorder is type II diabetes, type I diabetes, or a related disease leading to hyperglycemia or insulin tolerance.

In another embodiment of this aspect, the metabolic disease or disorder is type II diabetes or pre-type II diabetes.

In another embodiment of this aspect, the metabolic disease or disorder is type I diabetes.

In another embodiment of this aspect, the diabetes-related disease or disorder is selected from cardiovascular diseases, neurodegenerative disorders, atherosclerosis, hypertension, coronary heart diseases, cancer, alcoholic and non-alcoholic fatty liver diseases, dyslipidemia, nephropathy, retinopathy, neuropathy, diabetic heart failure, and cancer.

In another embodiment of this aspect, the diabetes-related disease is a neurodegenerative disease.

In another embodiment of this aspect, the neurodegenerative disease is amyotrophic lateral sclerosis, Parkinson's disease, or Alzheimer's disease.

In another embodiment of this aspect, the mitochondrial uncoupling agent is used as a veterinarian drug to treat diabetes or a diabetes-associated disease, and the subject is a mammalian animal.

In another embodiment of this aspect, the subject is a human.

In another embodiment of this aspect, the mitochondrial uncoupling agent is administered in combination with a second anti-diabetic agent.

In another embodiment of this aspect, the mitochondrial uncoupling agent is administered prior to administration of the second anti-diabetic agent.

In another embodiment of this aspect, the mitochondrial uncoupling agent is administered concomitantly with administration of the second anti-diabetic agent.

In another embodiment of this aspect, the mitochondrial uncoupling agent is administered subsequent to administration of the second anti-diabetic agent.

In another embodiment of this aspect, the second anti-diabetic agent is selected from insulin, insulin analogs, sulfonylureas, biguanides, meglitinides, thiazolidinediones, alpha glucosidase inhibitors, GLP-1 agonists, DPP-4 inhibitors.

In another embodiment of this aspect, the second anti-diabetic agent is metformin.

In another embodiment of this aspect, the mitochondrial uncoupling agent is administered orally, intravenously, or intraperitoneally.

In another aspect the present invention provides a method for long-term disease management of a metabolic disease or disorder, comprising administering to a subject in need of such long-term management an effective amount of a mitochondrial uncoupling agent selected from 2-hydroxy-benzoic anilide compounds, benzimidazoles, N-phenylanthranilates, phenylhydrazones, salicylic acids, acyldithiocarbazates, cumarines, and aromatic amines that have mitochondrial uncoupling activities, or a pharmaceutically acceptable salt, solvate, or prodrug thereof.

In another embodiment of this aspect, the metabolic disease or disorder is obesity, obesity-related complications.

In another embodiment of this aspect, the metabolic disease or disorder is type II diabetes or diabetes-related complications.

In another aspect the present invention provides use of a mitochondrial uncoupling agent in manufacture of a medicament for treatment or prevention of type II diabetes, obesity, or related disorders or complications.

In another aspect the present invention provides a compound of formula (I):

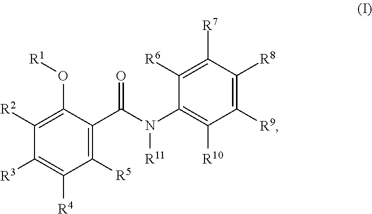

or a pharmaceutically acceptable salt, solvate, or prodrug thereof, wherein

R¹ and R¹¹ are each independently hydrogen (H) or a protecting group that can be hydrolyzed in vivo to become hydrogen;

R² through R⁵ are independent, at least one of which is selected from the group consisting of —OH, halogen, —CN, —NO₂, —CH(CH₃)₂, —C(CH₃)₃, and trihalo-methyl; and the rest of which are selected from —H, C₁₋₆alkyl, C₁₋₆alkenyl, C₁₋₆alkynyl, C₁₋₆alkoxy, C₁₋₆haloalkyl, hydroxyC₁₋₆alkyl, heteroaryl, and phenyl, wherein said heteroaryl or phenyl is optionally substituted with one to five substituents independently selected from Cl, Br, F, CF₃, and methoxy;

R⁶ through R¹⁰ are independent, at least one of which is selected from the group consisting of —OH, halogen, —CN, —NO₂, —CH(CH₃)₂, —C(CH₃)₃, and trihalo-methyl; and the rest of which are selected from —H, C₁₋₆alkyl, C₁₋₆alkenyl, C₁₋₆alkynyl, C₁₋₆alkoxy, C₁₋₆haloalkyl, hydroxyC₁₋₆alkyl, heteroaryl, and phenyl, wherein said heteroaryl or phenyl is optionally substituted with one to five substituents independently selected from Cl, Br, F, CF₃, and methoxy.

In one embodiment of this aspect, R¹ and R¹¹ are each independently hydrogen, —C(═O)NR^{a}R^{b}, or an acyl group independently selected from the group consisting of:

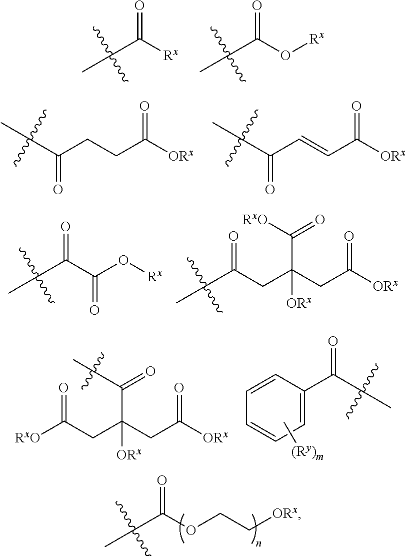

wherein m is 0, 1, 2, 3, 4, or 5;

n is an integer from 1 to 200;

R^{x} at each occurrence is independently hydrogen or C₁-C₈ alkyl; and

R^{y} at each occurrence is independently C₁-C₄ alkyl, halogen, hydroxyl, C₁-C₄ alkoxy, —NO₂, —CN, or —CO₂R^{x}.

In another embodiment of this aspect, R¹ is hydrogen or acetyl; R¹¹ is hydrogen; R² is hydrogen or methyl, R³ is hydrogen, R⁴ is Cl or Br, R⁵ is hydrogen, R⁶ is hydrogen, —Cl, —CH₃, or —NO₂, R⁷ is hydrogen or Cl, R⁸ is —H, —Cl, —NO₂, R⁹ is H, Cl or Br, and R¹⁰ is H or Cl.

In another aspect the present invention provides a composition for treatment or prevention of type II diabetes, obesity, a related disorder and complication, the composition comprising a compound of formula (I), or a pharmaceutically acceptable salt, solvate, or prodrug thereof, wherein:

R¹ and R¹¹ are each independently hydrogen (H) or a protecting group that can be hydrolyzed in vivo to become hydrogen;

R² through R⁵ are independent, at least one of which is selected from the group consisting of —OH, halogen, —CN, —NO₂, —CH(CH₃)₂, —C(CH₃)₃, and trihalo-methyl; and the rest of which are selected from —H, C₁₋₆alkyl, C₁₋₆alkenyl, C₁₋₆alkynyl, C₁₋₆alkoxy, C₁₋₆haloalkyl, hydroxyC₁₋₆alkyl, heteroaryl, and phenyl, wherein said heteroaryl or phenyl is optionally substituted with one to five substituents independently selected from Cl, Br, F, CF₃, and methoxy;

R⁶ through R¹⁰ independent, at least one of which is selected from the group consisting of —OH, halogen, —CN, —NO₂, —CH(CH₃)₂, —C(CH₃)₃, and trihalo-methyl; and the rest of which are selected from —H, C₁₋₆alkyl, C₁₋₆alkenyl, C₁₋₆alkynyl, C₁₋₆alkoxy, C₁₋₆haloalkyl, hydroxyC₁₋₆alkyl, heteroaryl, and phenyl, wherein said heteroaryl or phenyl is optionally substituted with one to five substituents independently selected from Cl, Br, F, CF₃, and methoxy.

In another embodiment of this aspect, the composition contains 2′,5-dichloro-4′-nitro salicylic anilide 2-aminoethanol salt (OSSA).

In another embodiment of this aspect, the composition further contains a pharmaceutically acceptable carrier.

In another aspect the present invention provides a method of treating or preventing a metabolic disease or disorder in a subject, comprising administering to the subject a therapeutically effective amount of a mitochondrial uncoupling agent or composition described above.

In another embodiment of this aspect, The method of claim 37, wherein the metabolic disease or disorder is type II diabetes, type I diabetes, or related diseases leading to hyperglycemia or insulin tolerance.

In another embodiment of this aspect, the metabolic disease or disorder is type II diabetes.

In another embodiment of this aspect, the diabetes-related disease or disorder is selected from cardiovascular diseases, neurodegenerative disorders, atherosclerosis, hypertension, coronary heart diseases, cancer, alcoholic and non-alcoholic fatty liver diseases, dyslipidemia, nephropathy, retinopathy, neuropathy, diabetic heart failure, and cancer.

In another embodiment of this aspect, the diabetes-related disease is a neurodegenerative disease.

In another embodiment of this aspect, the neurodegenerative disease is amyotrophic lateral sclerosis, Parkinson's disease, or Alzheimer's disease.

In another embodiment of this aspect, the subject is a mammalian animal.

In another embodiment of this aspect, the subject is a human.

In another embodiment of this aspect, the mitochondrial uncoupling agent is administered in combination with a second anti-diabetic agent.

In another embodiment of this aspect, the mitochondrial uncoupling agent is administered prior to administration of the second antidiabetic agent.

In another embodiment of this aspect, the mitochondrial uncoupling agent is administered concomitantly with administration of the second antidiabetic agent.

In another embodiment of this aspect, the mitochondrial uncoupling agent is administered subsequently to administration of the second antidiabetic agent.

In another embodiment of this aspect, the second anti-diabetic agent is selected from insulin, insulin analogs, sulfonylureas, biguanides, meglitinides, thiazolidinediones, alpha glucosidase inhibitors, GLP-1 agonists, and DPP-4 inhibitors.

In another embodiment of this aspect, the second anti-diabetic agent is metformin.

In another embodiment of this aspect, the mitochondrial uncoupling agent is administered orally, intravenously, or intraperitoneally.

In another aspect the present invention provides use of a compound of formula (I) described above as a mitochondrial uncoupling agent in manufacture of a medicament for treatment of diabetes, obesity, or related disorders or complications.

Thus, the present invention provides, among others, a method of treating and alleviating the symptoms of pre-type II diabetes (characterized by elevated blood glucose level) and complications of obesity or diabetes-related metabolic disorders, including, but not limited to, hypertension, cardiovascular diseases, nephropathy, and neuropathy. These diseases or disorders may be caused by dietary, environmental, medical and/or genetic factors. The method of the present invention can also be used for prevention of pre-type II diabetes and type II diabetes for a subject with risk factors including, but not limited to, obesity, dietary, and genetic predispositions and prevention of patients at risk from becoming obese and/or have obesity-related complications. In addition, the present invention provides a new approach for long-term chronic disease management and longevity management by reducing glucose levels in the blood.

In particular, the present invention provides a method of treating or alleviating the symptoms of type II diabetes, using the family of 2-hydroxy-benzoic anilide compounds and derivatives or related compounds. Representative examples of 2-hydroxy-benzoic anilide compounds are set forth in Table 1.

TABLE 1
Representative 2-hydroxy-benzoic anilide compounds used in the present
invention.

| No. | Name | R1 | R2 | R3 | R4 | R5 | R6 | R7 | R8 | R9 | R10 |
|---|---|---|---|---|---|---|---|---|---|---|---|
| 1 | 5,2′,5′ trichloro 4′ nitrosalicylic | H | H | H | Cl | H | Cl | H | NO₂ | Cl | H |
| | anilide | | | | | | | | | | |
| 2 | 5,2′ dichloro 4′ nitrosalicylic anilide | H | H | H | Cl | H | Cl | H | NO₂ | H | H |
| 3 | 5,3′,5′ trichloro 2′ nitro salicylic | H | H | H | Cl | H | NO₂ | Cl | H | Cl | H |
| | anilide | | | | | | | | | | |
| 4 | 5,2′,5′ trichloro 3 methyl 4′ nitro | H | CH₃ | H | Cl | H | Cl | H | NO₂ | Cl | H |
| | salicylic anilide | | | | | | | | | | |
| 5 | 5,5′ dichloro 2′ methyl 4′ nitro | H | H | H | Cl | H | CH₃ | H | NO₂ | Cl | H |
| | salicylic anilide | | | | | | | | | | |
| 6 | 5,4′ dichloro 2′ nitro salicylic anilide | H | H | H | Cl | H | NO₂ | H | Cl | H | H |
| 7 | 5,2′,5′ trichloro 4′ nitro 2 acetoxy | OCCH₃ | H | H | Cl | H | Cl | H | NO₂ | Cl | H |
| | benzanilide | | | | | | | | | | |
| 8 | 5,2′,5′ trichloro 3 methyl 4′ nitro 2 | OCCH₃ | CH₃ | H | Cl | H | Cl | H | NO₂ | Cl | H |
| | acetoxy benzanilide | | | | | | | | | | |
| 9 | 2′,5 dichloro 4′ nitro salicylic anilide | H | H | H | Cl | H | H | H | NO₂ | H | Cl |
| | (niclosamide) | | | | | | | | | | |
| 10 | 5 chloro salicyl 2 chloro 4 nitro | H | H | H | Cl | H | H | H | NO₂NH₂(CH₂)₂OH | H | Cl |
| | anilide 2 amino ethanol salt | | | | | | | | | | |
| 11 | 5 chloro salicyl 2 chloro 4 nitro | H | H | H | Cl | H | H | H | C₄H₁₀N₂ | H | Cl |
| | anilide pipe razine salt | | | | | | | | | | |
| 12 | 5 chloro salicyl 2 chloro 4 nitro | H | H | H | Cl | H | H | H | NO₂H₂O | H | Cl |
| | anilide monohydrate | | | | | | | | | | |
| 13 | 5,5′ dibromo salicyl | H | H | H | Br | H | H | H | NO₂ | Br | H |

Mitochondria are organelles in cells that are at the center of glucose and fatty acid metabolic pathways. They are the place where beta-oxidation of free fatty acids, citric acid cycle, and oxidative phosphorylation occur. The net effect of beta-oxidation, citric acid cycle, and oxidative phosphorylation are oxidation of pyruvate (from glycolysis of glucose) and fatty acids to produce carbon dioxide, water and the chemical energy for generation of a proton gradient across mitochondrial inner membrane. In turn, the proton influx across the mitochondrial membrane F₀-F₁-ATPase drives the formation of ATP molecules. The proton gradient across mitochondrial membrane can be dissipated, a process called mitochondrial uncoupling, which causes a futile cycle of oxidation of lipids or pyruvate (from glucose) without generating ATP.

Mitochondrial uncoupling can be induced by chemical uncouplers, for example, 2,4-dinitrophenol (DNP), which has various major side effects at higher doses, including causing hyperthermia. Among the first 100,000 persons treated with DNP, two fatalities occurred due to hyperthermia; therefore, DNP was withdrawn from the market.

Whether systemic treatment with chemical mitochondrial uncouplers can reduce blood glucose levels or increase insulin sensitivity, or whether chemical mitochondrial uncouplers can be used to treat type II diabetes remain unclear until the present invention. Moreover, the relative severe side effects observed from 2,4-dinitrophenol at high dosages had prevented one from attempting to use 2,4-dinitrophenol or any other mitochondrial uncouplers for the purpose of prevention and treatment of diabetes or diseases associated with diabetes.

Therefore, this invention was designed to search for safe chemical mitochondrial uncouplers and evaluate their efficacy in treating type II diabetes. We found that 5-chloro-salicyl-(2-chloro-4-nitro) anilide 2-aminoethanol salt (CSAA), a salt form of an FDA approved anthelmintic drug whose mechanism of action is uncoupling mitochondria in parasites, is highly effective in reducing fasting blood glucose and insulin concentrations, increasing glucose uptake, increasing insulin sensitivity, and reducing liver lipid load. Its limited solubility would prevent or eliminate the side effects observed with DNP at high dosages. It is expected that the derivatives of this compound, their prodrugs, or other mitochondrial uncouplers that have limited solubility would have a similar efficacy and safety profile.

In this invention we demonstrated that acute and chronic treatment with CSAA is efficacious in reducing plasma glucose concentrations in a diabetic mouse model. Moreover, we showed that chronic treatment with CSAA can prevent high-fat diet induced hyperglycemic condition, reduce fasting insulin levels, and increase insulin sensitivity. Our results support that the mechanism underlying the hypoglycemic effect of CSAA is mediated by its mitochondrial uncoupling activity. CSAA increases AMPK activities and increases glucose uptake in liver, muscles and other tissues. In vitro assay indicates that the concentrations that CSAA uncouples mitochondria in cultured cells are within the ranges of plasma CSAA levels upon oral administration. Importantly, alteration of the functional group in CSAA molecule that is essential for mitochondrial uncoupling totally abolishes its hypoglycemic effect in vivo. Together, our study not only provided strong data validating a potential new approach for preventing or treating hyperglycemia in type II diabetes, but also provided a good candidate molecule with excellent safety profile, which may be used for further development of new anti-diabetic drugs.

DNP is, in fact, not an efficient mitochondrial uncoupler, which functions at mini molar concentrations. DNP has side effects that are not only associated with its uncoupling activity at high dosages, such as hyperthermia, but also it has side effects that are specific for DNP. Fortunately, mitochondrial uncoupling activity turns out to be not inherently associated with severe adverse effects. This invention demonstrates that CSAA is a much more efficacious mitochondrial uncoupler. It functions at high nano molar to low micro molar concentrations. What really sets CSAA apart from DNP is that CSAA has very limited solubility in aqueous solution. This is likely the reason that niclosamide, the free base of CSAA and the pharmacophore of mitochondrial uncoupling activity of CSAA, has good safety profile and is an FDA approved anthelmintic drug. Due to the unique combination of pharmacodynamic and pharmacokinetic properties, CSAA family compounds have a good prospective for further development as oral anti-diabetic drugs.

Higher mitochondrial membrane potential is associated with increased production of mitochondrial reactive oxygen species (ROS). Mitochondrial ROS are important etiological factors for other pathological conditions, including aging, cancer, and neurodegenerative diseases. It is expected that CSAA and its derivatives would be effective in reducing mitochondrial ROS and might be useful for preventing and treating those diseases.

### Definitions

As used herein, the term “alkyl” is intended to include both branched and straight-chain saturated aliphatic hydrocarbon groups having the specified number of carbon atoms. For example, “C₁-C₁₀ alkyl” or “C₁₋₁₀ alkyl” (or alkylene), is intended to include C₁, C₂, C₃, C₄, C₅, C₆, C₇, C₈, C₉, and C₁₀ alkyl groups. Additionally, for example, “C₁-C₆ alkyl” or “C₁₋₆ alkyl” denotes alkyl having 1 to 6 carbon atoms. Alkyl group can be unsubstituted or substituted with at least one hydrogen being replaced by another chemical group. Examples of alkyl groups include, but are not limited to, methyl (Me), ethyl (Et), propyl (e.g., n-propyl and isopropyl), butyl (e.g., n-butyl, isobutyl, t-butyl), and pentyl (e.g., n-pentyl, isopentyl, neopentyl).

“Alkenyl” is intended to include hydrocarbon chains of either straight or branched configuration having the specified number of carbon atoms and one or more, preferably one to three, carbon-carbon double bonds that may occur in any stable point along the chain. For example, “C₂-C₆ alkenyl” or “C₂₋₆ alkenyl” (or alkenylene), is intended to include C₂, C₃, C₄, C₅, and C₆ alkenyl groups. Examples of alkenyl include, but are not limited to, ethenyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl, and 4-methyl-3-pentenyl.

“Alkynyl” is intended to include hydrocarbon chains of either straight or branched configuration having one or more, preferably one to three, carbon-carbon triple bonds that may occur in any stable point along the chain. For example, “C₂-C₆ alkynyl” is intended to include C₂, C₃, C₄, C₅, and C₆ alkynyl groups; such as ethynyl, propynyl, butynyl, pentynyl, and hexynyl.

The term “alkoxy” or “alkyloxy” refers to an —O-alkyl group. “C₁-C₆ alkoxy” or “C₁₋₆ alkoxy” (or alkyloxy), is intended to include C₁, C₂, C₃, C₄, C₅, and C₆ alkoxy groups. Examples of alkoxy groups include, but are not limited to, methoxy, ethoxy, propoxy (e.g., n-propoxy and isopropoxy), and t-butoxy.

The term “halo” or “halogen” refers to fluoro, chloro, bromo, and iodo. “Haloalkyl” is intended to include both branched and straight-chain saturated aliphatic hydrocarbon groups having the specified number of carbon atoms, substituted with one or more halogens. Examples of haloalkyl include, but are not limited to, fluoromethyl, difluoromethyl, trifluoromethyl, and trichloromethyl.

“Haloalkoxy” or “haloalkyloxy” represents a haloalkyl group as defined above with the indicated number of carbon atoms attached through an oxygen bridge. For example, “C₁-C₆ haloalkoxy” or “C₁₋₆ haloalkoxy”, is intended to include C₁, C₂, C₃, C₄, C₅, and C₆ haloalkoxy groups. Examples of haloalkoxy include, but are not limited to, trifluoromethoxy, trichloromethoxy, and 2,2,2-trifluoroethoxy.

“Aryl” groups refer to monocyclic or polycyclic aromatic hydrocarbons, including, for example, phenyl, and naphthyl. “C₆-C₁₀ aryl” or “C₆₋₁₀ aryl” refers to phenyl and naphthyl. Unless otherwise specified, “aryl”, “C₆-C₁₀ aryl,” “C₆₋₁₀ aryl,” or “aromatic residue” may be unsubstituted or substituted with 1 to 5 groups selected from —OH, —OCH₃, —Cl, —F, —Br, —I, —CN, —NO₂, —NH₂, —NH(CH₃), —N(CH₃)₂, —CF₃, —OCF₃, —C(O)CH₃, —SCH₃, —S(O)CH₃, —S(O)₂CH₃, —CH₃, —CH₂CH₃, —CO₂H, and —CO₂CH₃. The term “benzyl,” as used herein, refers to a methyl group on which one of the hydrogen atoms is replaced by a phenyl group, wherein said phenyl group may optionally be substituted by one to five, preferably one to three, substituents independently selected from methyl, trifluoromethyl (—CF₃), hydroxyl (—OH), methoxy (—OCH₃), halogen, cyano (—CN), nitro (—NO₂), —CO₂Me, —CO₂Et, and —CO₂H. Representative examples of benzyl group include, but are not limited to, PhCH_{2—, 4}-MeO—C₆H₄CH₂—, 2,4,6-tri-methyl-C₆H₂CH₂—, and 3,4-di-Cl-C₆H₃CH₂—.

As used herein, the term “heteroaryl” is intended to mean stable monocyclic and polycyclic aromatic hydrocarbons that include at least one heteroatom ring member, such as sulfur, oxygen, or nitrogen. Heteroaryl groups include, without limitation, pyridyl, pyrimidinyl, pyrazinyl, pyridazinyl, triazinyl, furyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, benzodioxolanyl, and benzodioxane. Heteroaryl groups are substituted or unsubstituted. The nitrogen atom is substituted or unsubstituted (i.e., N or NR wherein R is H or another substituent, if defined). The nitrogen and sulfur heteroatoms may optionally be oxidized (i.e., N→O, and S(O)ₚ, wherein p is 0, 1 or 2).

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18^{th} Edition, Mack Publishing Company, Easton, Pa., 1990, the disclosure of which is hereby incorporated by reference.

In addition, compounds of Formula (I) may have prodrug forms. Any compound that will be converted in vivo to provide the bioactive agent (i.e., a compound of Formula (I) is a prodrug within the scope and spirit of the invention. Various forms of prodrugs are well known in the art. For examples of such prodrug derivatives, see:

a) Design of Prodrugs, edited by H. Bundgaard (Elsevier, 1985), and Methods in Enzymology, Vol. 112, at pp. 309-396, edited by K. Widder et al. (Academic Press, 1985);

b) A Textbook of Drug Design and Development, edited by Krosgaard-Larsen and H. Bundgaard, Chapter 5, “Design and Application of Prodrugs,” by H. Bundgaard, at pp. 113-191 (1991);

Preparation of prodrugs is well known in the art and described in, for example, Medicinal Chemistry: Principles and Practice, F. D. King, ed., The Royal Society of Chemistry, Cambridge, UK, 1994; Hydrolysis in Drug and Prodrug Metabolism. Chemistry, Biochemistry and Enzymology, B. Testa, J. M. Mayer, VCHA and Wiley-VCH, Zurich, Switzerland, 2003; The Practice of Medicinal Chemistry, C. G. Wermuth, ed., Academic Press, San Diego, Calif., 1999.

The compounds of the present invention may be prepared by the exemplary processes described in relevant published literature procedures that are used by one skilled in the art.

### EXAMPLES

The present invention is described more fully by way of the following non-limiting examples. Modifications of these examples will be apparent to those skilled in the art.

### Material and Methods

### Mouse and Treatment

The 5-week old db/db mice and the C57/B16 mice were purchased from Jackson Laboratory and housed in the vivarium of UMDNJ-RWJMS. Starting at the age of 6 weeks the db/db mice were either fed with normal AIN-93M (Research Diet) or with AIN-93M diet containing 1500 ppm CSAA. For the C57/B16 mice, at the age of 5 weeks, the mice were either fed with high-fat diet (60% fat calorie, Research Diet), or with high-fat diet containing 1500 ppm CSAA. For acute I.P. injection, mice were starved for 5 hours, CSAA or the sulfite conjugated CSAA (customer synthesized by Provid Inc.) were dissolved in saline or saline containing 10% DMSO. A total volume of 100 micro liter solution (with or without 100 microgram CSAA or its derivative) was injected in each mouse. For food intake experiments, sets of mice were housed individually in metabolic cages (Nalgene) with free access to food and water. Food cups and food scattered in the runway to the cups were weighed daily to determine food intake. For mouse tissue studies, mice were sacrificed by decapitation, and the tissues of interesting were obtained.

### Measurement of Blood Glucose and Insulin

Blood glucose was determined using OneTouch UltraSmart blood glucose monitoring system (Lifescan), and the insulin levels were measured by ultra sensitive mouse insulin ELISA kit (Crystal Chem Inc.), following the manufacturer's instructions.

### Glucose Tolerance Assay and Insulin Tolerance Assay

For glucose tolerance tests, mice were starved overnight and injected I.P. with 20% glucose at a dose of 2 g/kg body weight. Blood was obtained from the tail at time points 0, 15, 30, 60, 90, and 120 min for glucose measurement. For insulin tolerance tests, mice were starved for 5 h and injected I.P. with 0.75 U/kg body weight recombinant human insulin (Eli Lilly). Blood was obtained from the tail at time points 0, 15, 30, 60, 90, and 120 min for glucose measurement.

### Immunoblotting Assay and Mouse Liver Histological Analysis

Immunoblotting assays were carried out according to standard protocol. The sources of the antibodies are: AMPK antibody (Cell Signaling Technology), Thr-172-phosphorylated AMPK antibody (Cell Signaling Technology), Ran antibody (C-20, Santa Cruz).

For mouse liver histological study, the mice sacrificed by decapitation. Liver slices were fixed with buffered formalin (Surgipath Medical Industries, Inc.) and embedded in paraffin. Tissue slides were stained with hematoxylin and eosin (H&E) for detection of lipid droplets in tissue samples. Pictures were taken with a Universal Microscope Axioplan 2 imaging system (Carl Zeiss) with phase contrast objectives.

Glucose Uptake Assay with ³H 2-deoxyglucose

Mice were starved for 3 hours, followed by treatment with saline or saline containing CSAA through I.P. injection at the dosage of 100 microgram/mouse. 1.5 hours later, ³H-2-deoxyglucose (0.5 microcurrie/gram of body weight) was injected through I.P. route. 30 minutes later, mice were treated with anesthetics and perfused with PBS. Mice were then sacrificed and tissue was extracted for measurement of ³H-2-deoxyglucose accumulation (normalized against tissue mass).

### Mitochondrial Uncoupler Activity Assay

For mitochondrial uncoupling assay with isolated mitochondria: Mitochondria were isolated from mouse liver. 1.0 mg of mitochondria in a volume of 0.9 ml of respiration buffer was analyzed for oxygen consumption in the presence of mitochondrial substrate as well as the various inhibitors with an Oxygraph System (Hansatech Instrument, Norfolk, UK). The final concentrations of the various chemicals added into the respiration buffer are as follows: succinate, 5 mM; ADP, 125 μM; oligomycin, 5 μg/ml; KCN, 2 mM)

For mitochondrial uncoupling activity analysis with the cultured cells, the NIH-3T3 were culture to 90% confluence. The cells were then treated with CSAA at various concentrations and for various periods of time. The cells were then treated with TMRE (Tetramethylrhodamine ethyl ester perchlorate) to a final concentration of 100 nM, incubate for 15 minutes. The cells were then washed twice with PBS, and the pictures were taken under microscope.

BD™ Oxygen Biosensor System was used to measure the cellular oxygen consumption. NIH-3T3 cells were cultured overnight to log phase, then seeded in oxygen biosensor 96-well plate at the density of 20000 cells per well in DMEM medium. Treatments were initiated by adding indicated drug CSAA (1 μM) or oligomycin (5 μg/ml), or both into the medium. Oxygen consumptions (decrease in oxygen concentrations) were indicated by generation of fluorescent signals which were initially quenched by oxygen. Cellular ATP concentrations were measured with the ENLITEN® ATP Assay System and normalized against cell number.

### Example 1

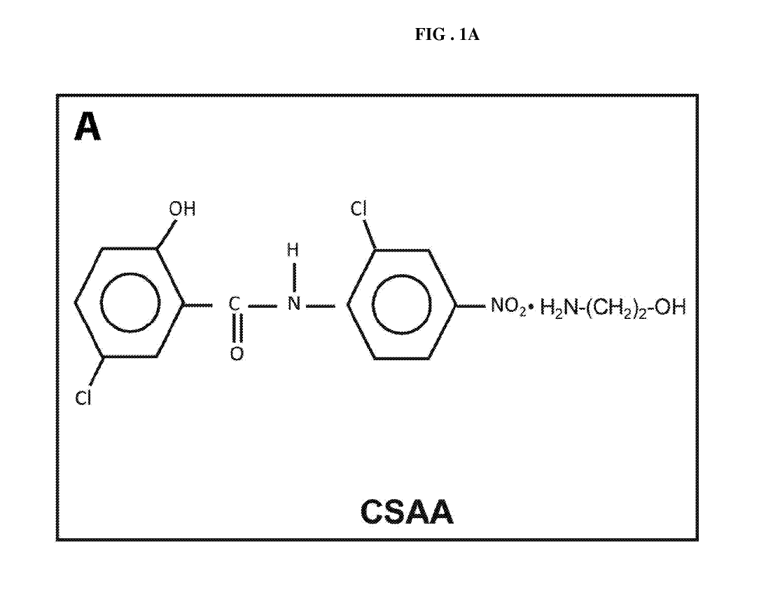

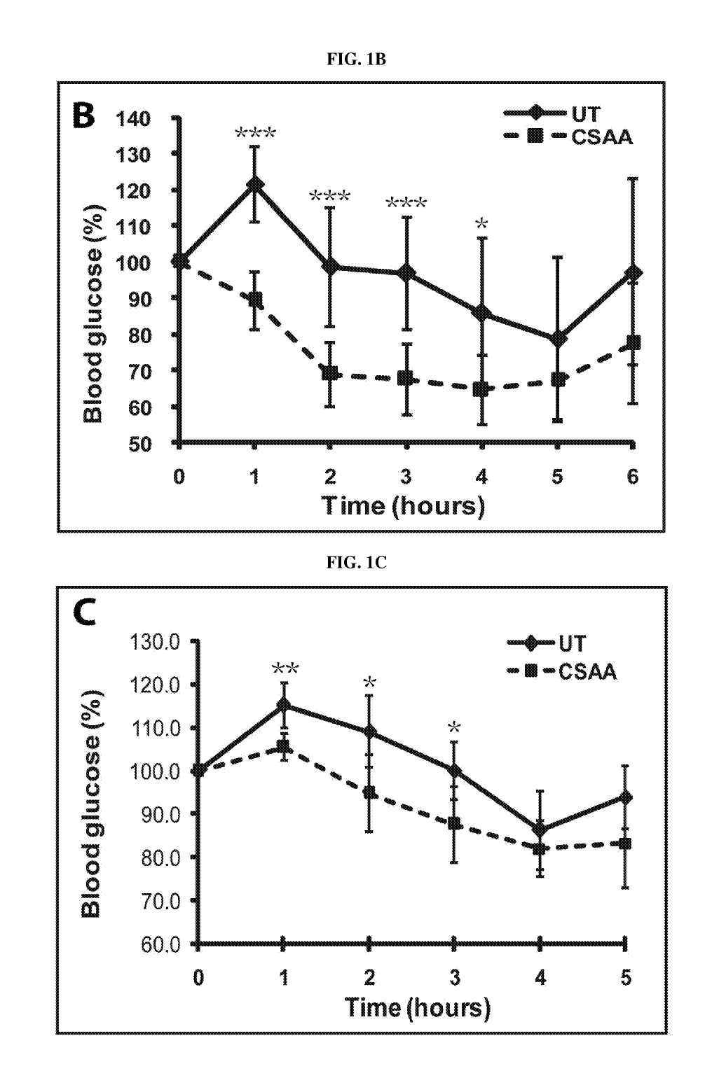

Acute treatment with 5-chloro-salicyl-(2-chloro-4-nitro) anilide 2-aminoethanol salt (CSAA) effectively reduces blood glucose in diabetic and pre-diabetic mice. The structure of CSAA is shown is FIG. 1A. To evaluate the effect of CSAA on blood glucose control, we injected the CSAA containing saline solution into either the diabetic db/db mice or the pre-diabetic C57/B16 mice fed with high-fat diet for 10 weeks. As shown in FIG. 1, treatment with CSAA lowered blood glucose concentrations ˜1 hours after treatment and remained effective until about 4 hours. The dramatic decrease in blood glucose concentration was observed in both mouse strains. It was particular significant in the db/db mice, which showed a 30% reduction in blood glucose as compared to the control. These results indicate that CSAA has an acute effect in reducing blood glucose concentrations in diabetic and pre-diabetic mouse models.

### Example 2

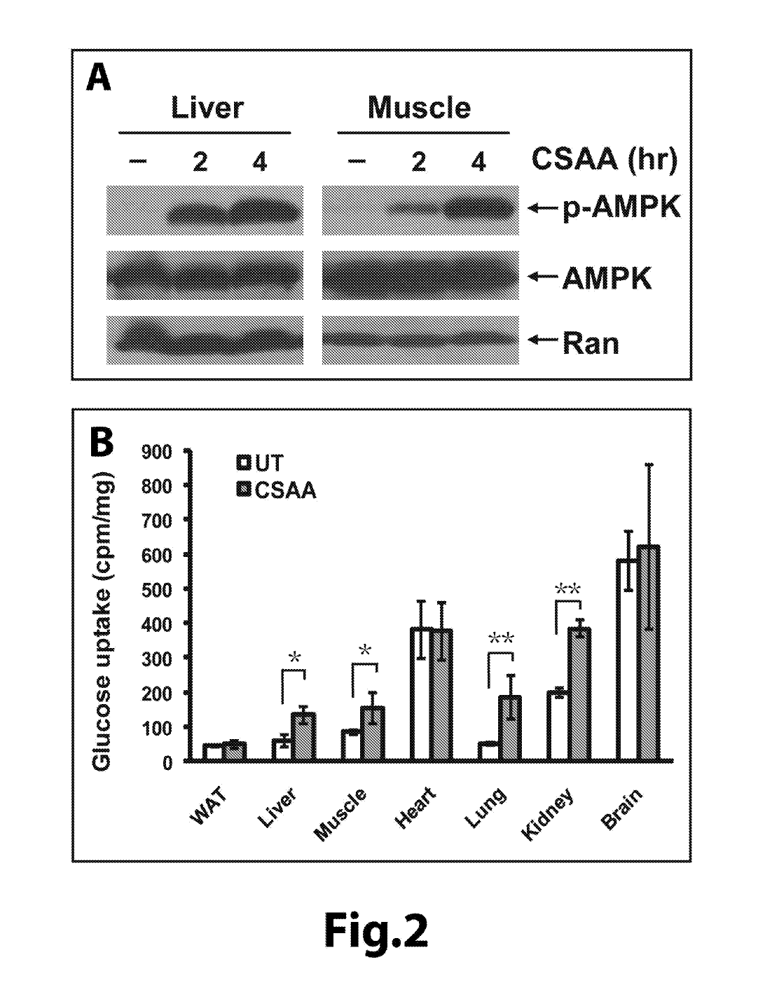

The acute effect of CSAA in reducing blood glucose is associated with AMP-activated kinase (AMPK) activation and increased glucose uptake in tissues. Mitochondrial uncouplers may decrease the efficiency of ATP production, which may in turn induce a compensatory upregulation of glucose uptake. To test this idea, we measured the levels of phosphorylated AMPK, which reflect the activity of AMPK, in mouse liver after CSAA injection. AMPK was activated in response to increase of AMP (adenosine monophosphate) as a result of reduction of ATP. As shown in FIG. 2A, indeed, acute treatment with CSAA dramatically increased the levels of the phosphorylated AMPK. In addition, we directly measured the glucose uptake rates in various tissues of the mice acutely treated with CSAA. As shown in FIG. 2B, the glucose uptake rates significantly increased in liver, muscles, kidney, and lungs, but not in brain or white adipose tissue (WAT). Together, these results indicate that the acute effect of CSAA in reducing blood glucose is likely mediated by its activity in reducing energy efficiency and consequent compensatory upregulation in glucose uptake.

### Example 3

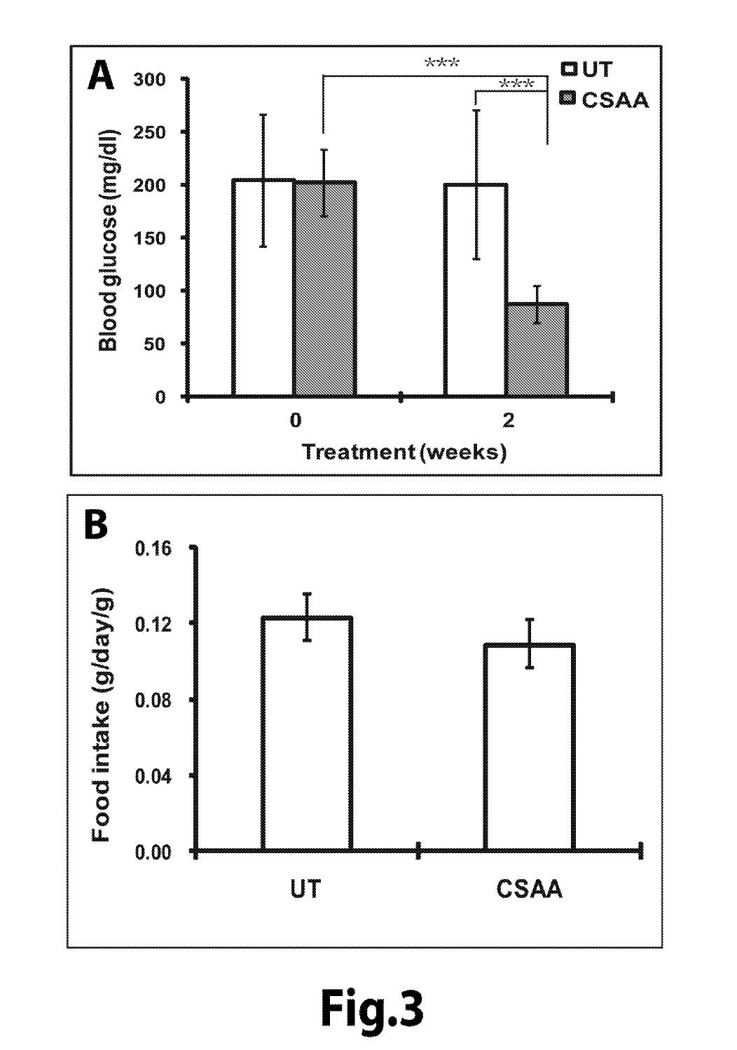

Chronic oral treatment with CSAA reduces fasting blood glucose concentrations in db/db diabetic mice. We further determined if chronic oral treatment with CSAA has beneficial effect in lowing blood glucose concentrations in the db/db diabetic mice. As shown in FIG. 3A, starting at the age of 6 weeks, a two week oral treatment of CSAA (by feeding the mice with food containing CSAA), reduced the fasting blood glucose of the db/db mice to almost normal levels. The food intake rates between the two groups (mice fed with normal food or food containing CSAA) were not significantly different (FIG. 3B), which ruled out the possibility that CSAA may affect appetite and food uptake thereby causing the hypoglycemic effect. As time went by, the fasting blood glucose levels of the CSAA treated mice went up. But they remained significantly lower than those in the non-CSAA treated mice (data not shown). Importantly, the body weight and adiposity of the CSAA-treated db/db mice were not different from the control mice (data not shown), indicating that the anti-diabetic effects of CSAA were not mediated through reducing the degree of obesity. This result indicates a remarkable efficacy of long-term oral CSAA treatment in improving glycemic control in the diabetic mouse model.

### Example 4

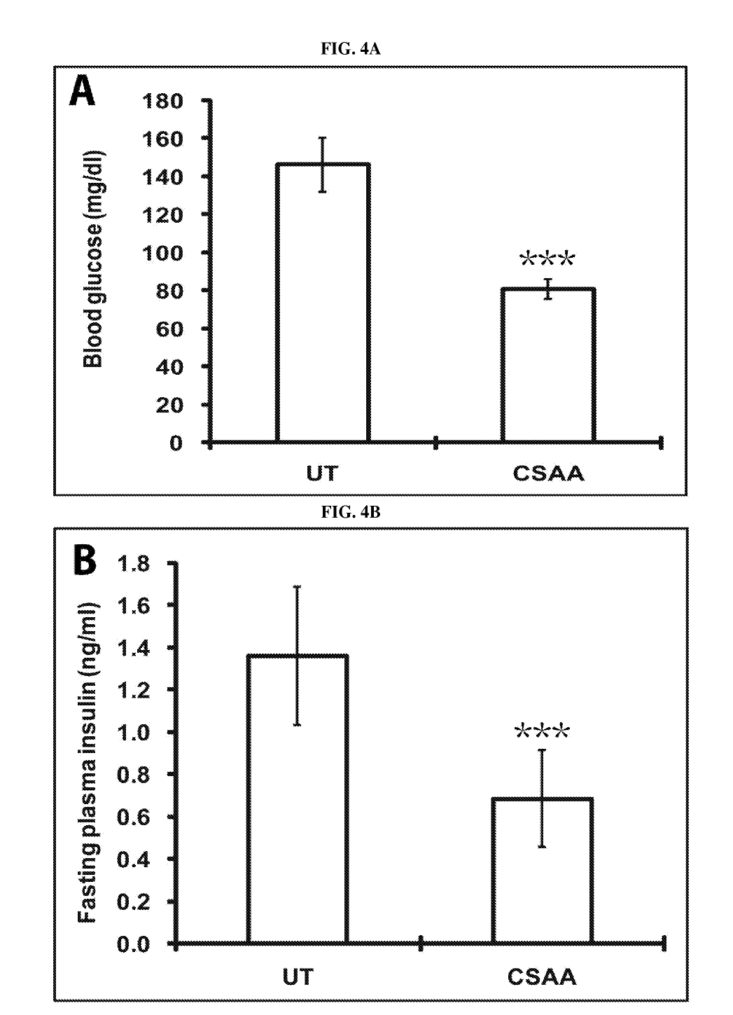

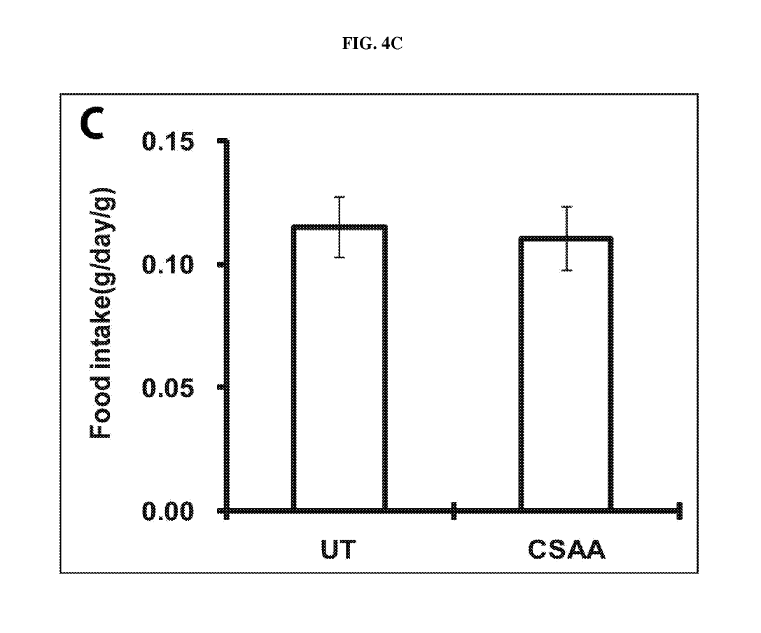

Chronic oral treatment with CSAA reduces fasting blood glucose and insulin concentrations in high-fat diet induced pre-diabetic mice. We then investigated the effect of chronic oral treatment with CSAA in the high-fat diet induced pre-diabetic mice. As shown in FIG. 4, feeding the C57/B16 mice with high-fat diet for 8 weeks induced a pre-diabetic condition by dramatically increasing the fasting blood glucose concentrations; while feeding the mice with high-fat diet containing CSAA completely prevented the increase of fasting blood glucose (FIG. 4A). Consistent with a better glycemic control, the fasting insulin concentrations in the CSAA treated group were significantly lower than those in the control group (FIG. 4B). Again, CSAA has no effect in food uptake in the mice (FIG. 4C). Moreover, the body weight and adiposity of the CSAA-treated mice were not different from the control mice (data not shown), again indicating that the anti-diabetic effects of CSAA were not mediated through reducing the degree of obesity. Together, these results indicate that CSAA can be highly effective in preventing diabetic conditions induced by high fat diet.

### Example 5

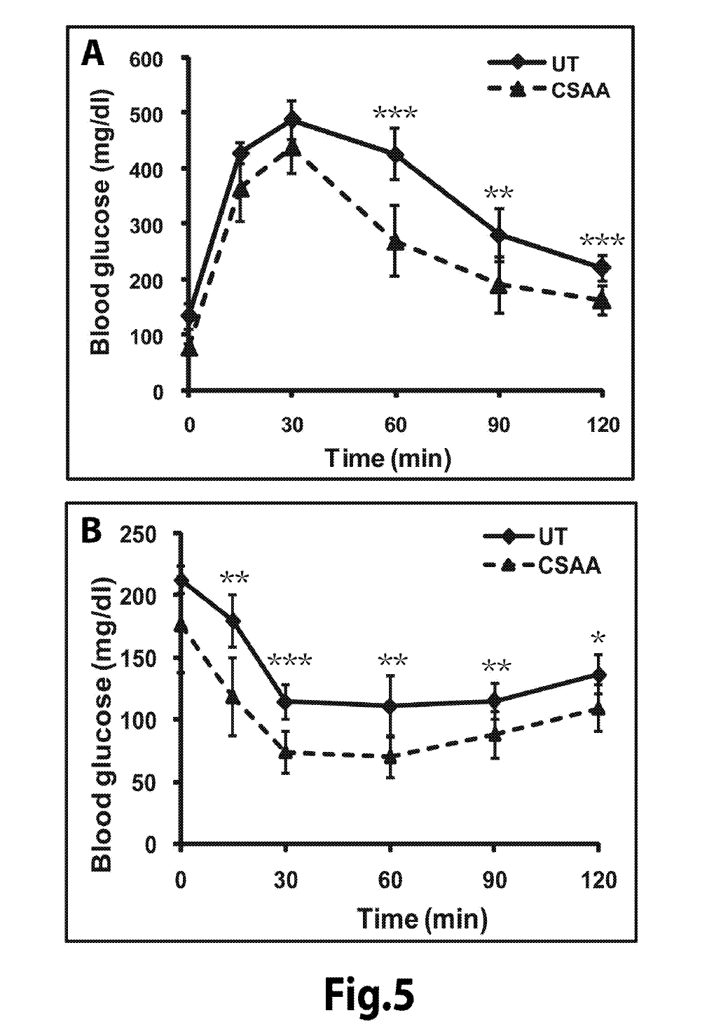

Chronic oral CSAA treatment increases insulin sensitivity. We analyzed the C57/B16 mice that were either fed with high-fat diet alone or high-fat diet mixed with CSAA in terms of insulin tolerance. As shown in FIG. 5, the CSAA fed mice exhibited significantly increased insulin sensitivity as measured either by glucose tolerance assay or by insulin tolerance assay. These results indicate long-term CSAA treatment can increase insulin sensitivity. This effect may have contributed to the dramatically reduced fasting glucose and insulin levels observed in the FIG. 4.

### Example 6

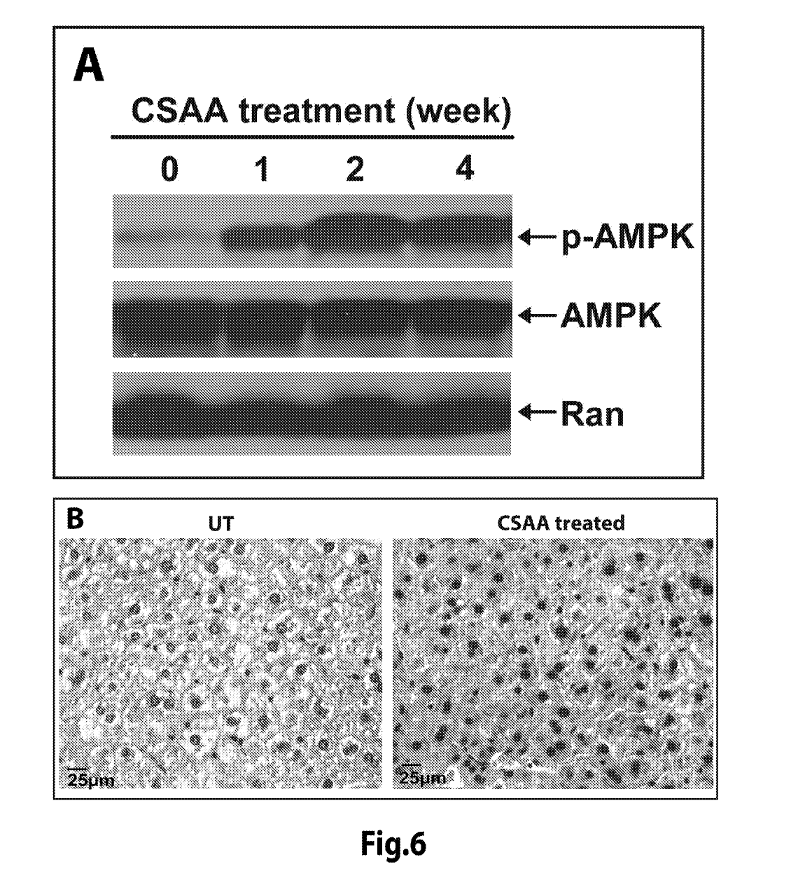

Chronic oral CSAA treatment increases tissue AMPK activity and reduces liver lipid load. To understand the mechanism by which CSAA reduces fasting glucose concentration and improves insulin sensitivity, we measured the effect of chronic oral CSAA treatment in liver AMPK activation and liver lipid accumulation in mice under high-fat diet. As shown in FIG. 6A, oral CSAA treatment chronically elevated the AMPK activity in liver. In addition, oral CSAA dramatically reduced the lipid load in hepatocytes (FIG. 6B). These results are consistent with the idea that the hypoglycemic effect of CSAA may relate to its acute effect in reducing cellular ATP levels thus increasing glucose uptake, as well as to its long-term effect in reducing lipid accumulation in peripheral organs such as liver, which increases insulin sensitivity.

### Example 7

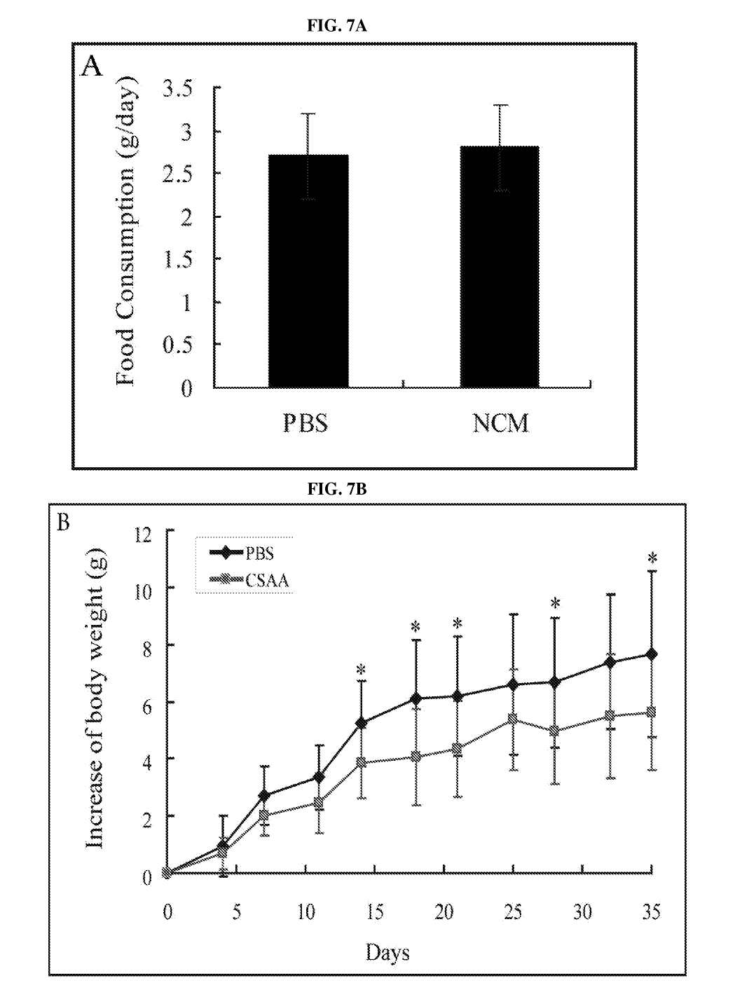

Chronic CSAA treatment via I.P. injection reduces high-fat induced weight gain. Those mice that were treated with CSAA via chronic oral administration as described from FIG. 2 to FIG. 6 exhibited significantly improved glycemic control, yet they did not differ from the untreated control mice in terms of body weight and adiposity. These results indicate that the anti-diabetic effects of CSAA in these mice were not mediated by reducing the levels of obesity. We ask a separate question, whether CSAA treatment can have impact on adiposity. To increase the bio-availability of CSAA, we performed chronic CSAA treatment via I.P. injection and the effect of CSAA on high-fat diet induced body weight gain was examined, 24 normal mice at the age of 8 months were fed with high-fat diet. Half of them (12 mice) were treated with daily CSAA injections via I.P. route at the dosage of 100 μg/mouse (in 500 μl PBS). The other 12 mice were injected daily with vehicle only (PBS). The body weight was measured twice a week and the net body weight gain for each mouse was determined. The average weight gain in each group over the experimental period was plotted. As shown in FIG. 7B, CSAA reduces the weight gain induced by high-fat diet. To rule out the possibility that CSAA affects body weight gain by reducing appetite, the food uptake rate of each mouse was also determined. The food uptake levels were measured daily with metabolic cages from the day 15 to day 29 and the average daily food uptake of each group was calculated. As shown in FIG. 7A, the CSAA -treated group actually had higher food uptake rate. These results indicate that CSAA treatment can have impact on adiposity if administrated at higher dosage and/or via a different and more effective administration route.

### Example 8

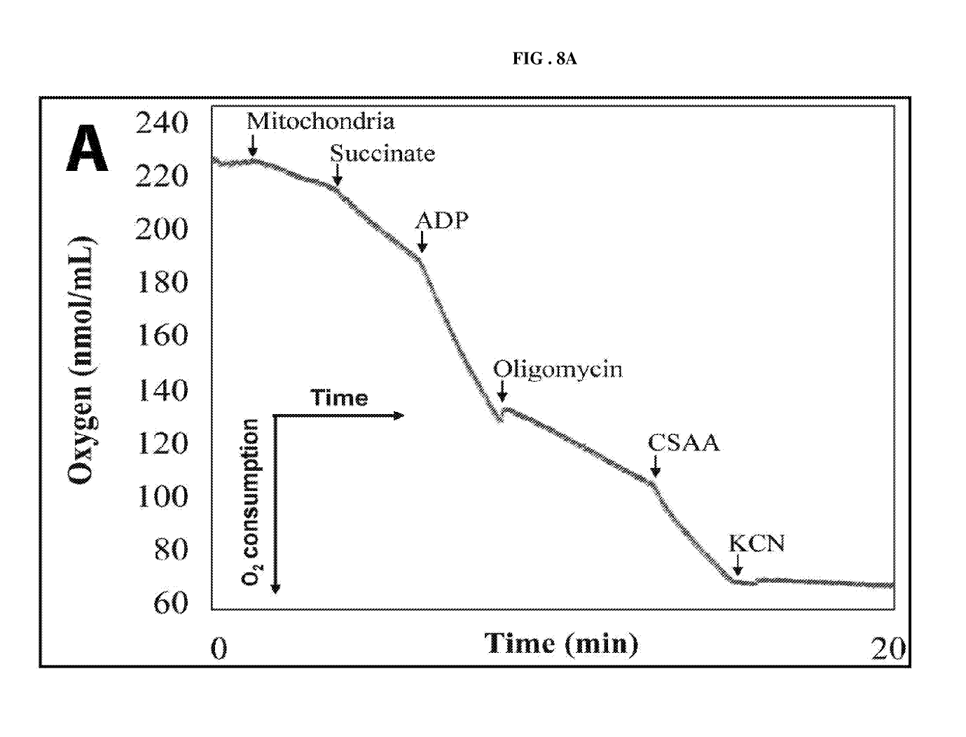

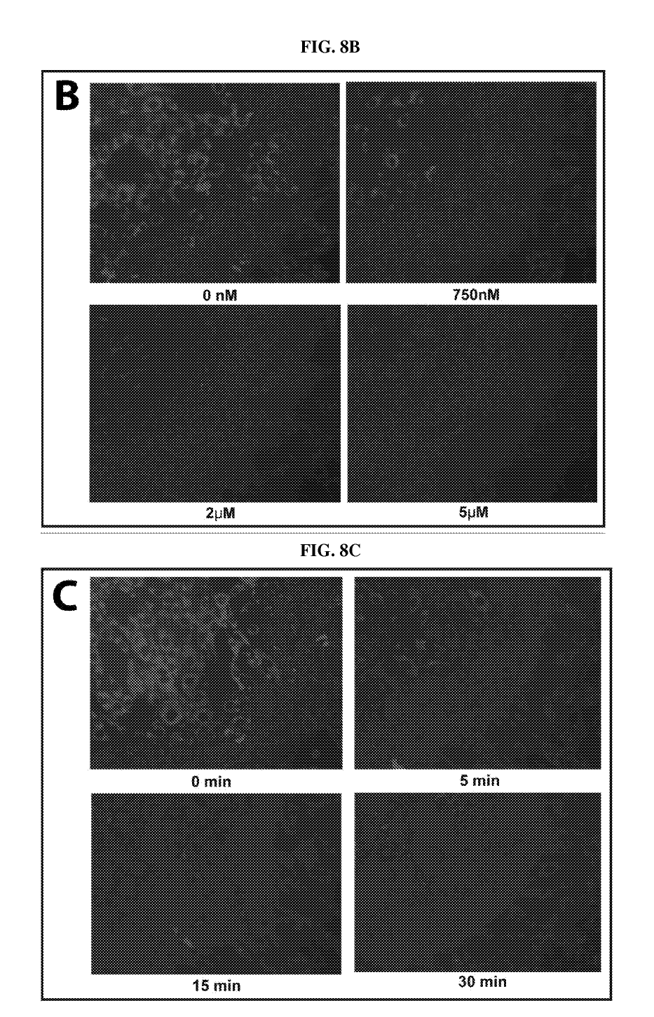

CSAA causes mitochondrial uncoupling at high nanomolar concentrations in cultured mammalian cells. Niclosamide, the free base of CSAA, is an FDA approved anthelmintic drug. The mechanism of action of niclosamide is uncoupling mitochondria in roundworms and other parasites in intestine. Niclosamide is extremely insoluble in aqueous solution, which is probably responsible for its low systemic bioavailability and excellent safety profile. The water solubility of CSAA is about 30 to 50 fold higher than niclosamide fee base, with a maximal plasma concentration at around 0.75˜2.0 micromolar after oral administration (0.25 to 0.60 mg/L) (Chemical Safety Information from Intergovernmental Organizations, WHO/VBC/DS/8863: WORLD HEALTH ORGANIZATION FOOD AND AGRICULTURE ORGANIZATION, 1988. http://www.inchem.org/documents/pds/pds/pest63_e.htm). We then determined the concentrations at which CSAA exhibits mitochondrial uncoupling activity with cultured mouse fibroblast cells. As shown in FIG. 8A, we first confirmed that CSAA has mitochondrial uncoupling activity on mammalian mitochondria isolated from mouse liver. In the presence of oligomycin, which inhibits F₀F₁-ATPase, CSAA still effectively promoted mitochondrial oxygen consumption, a feature that is unique to mitochondrial uncoupler. When analyzed with intact cells, as shown in FIG. 8B, CSAA exhibited activity in reducing mitochondrial membrane potential starting at the concentration of 500 nM. Full uncoupling of mitochondria could be seen at concentrations around 5 micromolar. Moreover, the action of CSAA in dissipating mitochondrial membrane potential was rapid, and its effect could be seen as early as 5 minutes after CSAA application (FIG. 8C), suggesting that the uncoupling activity was likely to be a direct and primary effect of CSAA. Together, the results indicate that CSAA can uncouple mitochondria at the concentrations of high nanomolar to low micromolar range in cultured cells, which is within the documented plasma concentration ranges of CSAA upon oral administration.

### Example 9

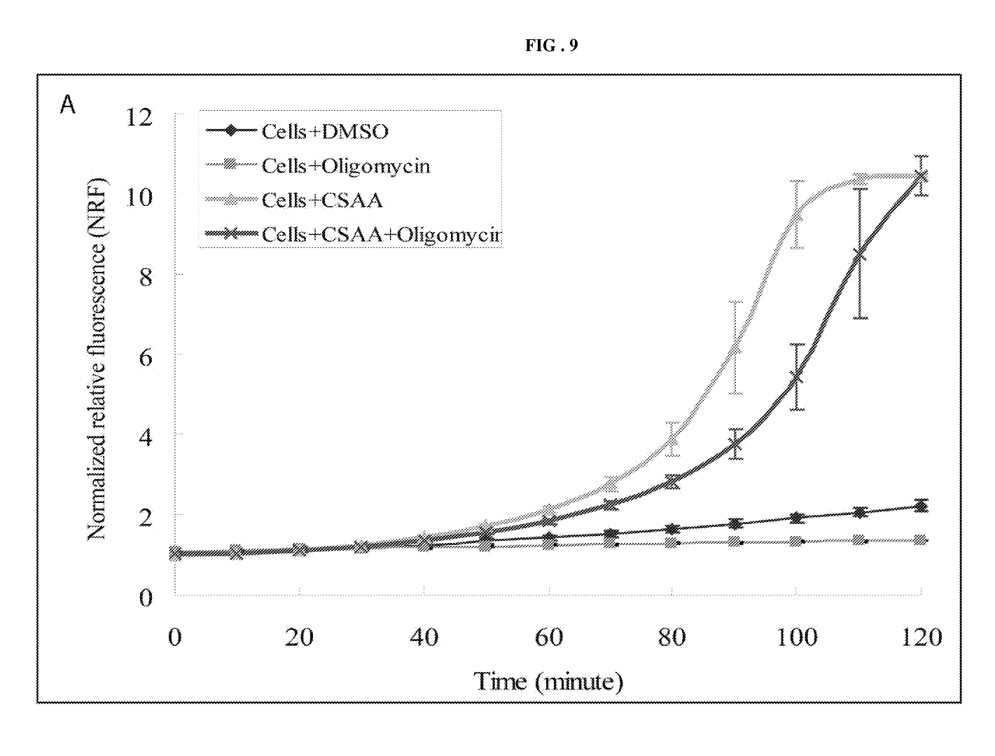

CSAA stimulates cellular oxygen consumption with or without co-treatment of oligomycin and does not affect steady-state ATP concentrations in cells. To further demonstrate that CSAA uncouples mitochondria at low micro molar concentrations in living cells and to rule out the possibility that the reduction of mitochondrial membrane potential as observed in FIG. 8 is due to loss of mitochondrial integrity, we measured oxygen consumption of the intact cells upon treatment with CSAA in the presence or absence of oligomycin. As shown in FIG. 9A, CSAA dramatically stimulated cellular oxygen consumption, indicating the function of the mitochondrial electron transport chain was normal and was activated by CSAA. Moreover, co-treatment with oligomycin, an inhibitor of the F₀F₁ ATPase, did not significantly affect CSAA-stimulated oxygen consumption. This directly demonstrates that CSAA is efficacious in uncoupling mitochondria in living cells at this concentration (1 μM). Despite the dramatically increased rates of mitochondrial oxidation as indicated by oxygen consumption, the cellular ATP concentrations did not increase (FIG. 9B). Nor did the ATP concentrations significantly decrease upon CSAA treatment. Likely, some compensatory responses such as increased glucose uptake and elevated rates of glycolysis might have contributed to the relatively stable intracellular ATP concentrations in the presence of CSAA.

### Example 10

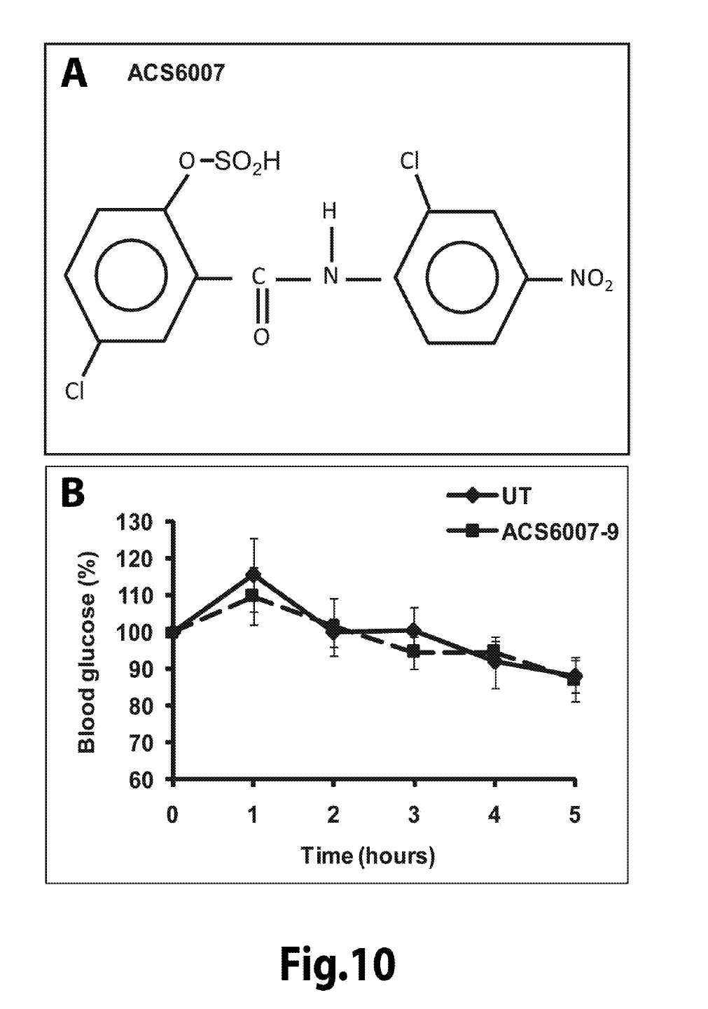

Changing the function group of CSAA that is essential for mitochondrial uncoupling activity abolishes the hypoglycemic effect. The various studies as shown in the previous sections strongly suggest that the uncoupling activity of CSAA mediated its anti-diabetic effect. To further demonstrate that the hypoglycemic effect of CSAA is related to mitochondrial uncoupling, we synthesized a derivative of CSAA, in which the 2-OH group was altered to a 2-O—SO₂H group (FIG. 10A). As well-established before, the mitochondrial uncoupling activity absolutely requires the 2-OH group, which is essential to make the molecule a highly lipophilic weak acid that shuffles proton across the mitochondrial inner membrane (Terada, H., Environ. Health Perspect. 1990, 87:213-218). Alteration of this structure to a polar sulfite group would destroy this property and abolish its mitochondrial uncoupling activity. We then tested if the sulfite derivative of CSAA is efficacious in reducing blood glucose. As shown in FIG. 10B, this modification at the 2-OH functional group totally abolished the hypoglycemic activity of CSAA. This result further demonstrates that indeed the mitochondrial uncoupling activity of CSAA is responsible for the anti-diabetic effect observed in this study.

The foregoing examples and description of the preferred embodiments should be interpreted as illustrating, rather than as limiting the present invention as defined by the claims. All variations and combinations of the features above are intended to be within the scope of the following claims.

1.-50. (canceled)

51. A method of treating or preventing a metabolic disease or disorder in a subject, comprising administering to the subject a therapeutically effective amount of 2′,5-dichloro-4′-nitro salicylic anilide (niclosamide), or a pharmaceutically acceptable salt thereof.

52. The method of claim 51, wherein the 2′,5-dichloro-4′-nitro salicylic anilide is 2′,5-dichloro-4′-nitro salicylic anilide 2-aminoethanol salt (CSAA).

53. The method of claim 51, wherein the metabolic disease or disorder is related to body-weight control.

54. The method of claim 51, wherein the metabolic disease or disorder is selected from obesity, obesity-related complications, hypertension, cardiovascular disease, nephropathy and neuropathy.

55. The method of claim 51, wherein the metabolic disease or disorder is related to elevated plasma glucose concentrations.

56. The method of claim 51, wherein the metabolic disease or disorder is type II diabetes, pre-type II diabetes, type I diabetes, or a related diabetes-related disease or disorder leading to hyperglycemia or insulin tolerance.

57. The method of claim 56, wherein the diabetes-related disease or disorder is selected from cardiovascular diseases, neurodegenerative disorders, atherosclerosis, hypertension, coronary heart disease, alcoholic and non-alcoholic fatty liver diseases, dyslipidemia, kidney failure, gangrene, nephropathy, retinopathy, neuropathy, gastrointestinal disorders, diabetic heart failure and cancer.

58. The method of claim 57, wherein the diabetes-related disease or disorder is a neurodegenerative disease.

59. The method of claim 58, wherein the neurodegenerative disease is amyotrophic lateral sclerosis, Parkinson's disease, or Alzheimer's disease.

60. The method of claim 51, wherein the subject is a mammalian animal.

61. The method of claim 51, wherein the subject is human.

62. The method of claim 51, wherein the 2′,5-dichloro-4′-nitro salicylic anilide, or a pharmaceutically acceptable salt thereof, is administered orally, intravenously, or intraperitoneally.

63. A method for long term disease management of a metabolic disease or disorder comprising administering chronically to a subject in need of such long term management a therapeutically effective amount of 2′,5-dichloro-4′-nitro salicylic anilide (niclosamide), or a pharmaceutically acceptable salt thereof, wherein the chronic administration effectuates at least one of increased insulin sensitivity, reduced plasma glucose levels, reduced plasma insulin levels, diminished lipid load in liver or muscle, or reduction of weight gain.

64. The method of claim 63, wherein the chronic administration to the subject does not substantially reduce at least one of weight, adiposity, or appetite.

65. A therapeutic composition for treating or preventing a metabolic disease or disorder in a subject comprising a pharmaceutically acceptable carrier, a therapeutically effective amount of 2′,5-dichloro-4′-nitro salicylic anilide (niclosamide), or a pharmaceutically acceptable salt thereof, and wherein the composition is effective for treating metabolic disease or disorder

66. The therapeutic of claim 65, wherein the composition comprises an effective amount of at least one other anti-diabetic therapeutic or another mitochondrial uncoupler.

67. The therapeutic composition of claim 66, wherein the anti-diabetic therapeutic is selected from the group consisting of metformin, sulfonurea, thiazolidinediones, glucosidase inhibitors, GLP-1 analogs, amylin analogs, DPP-4 inhibitors, insulin, and combinations thereof.

68. The therapeutic composition of claim 66, wherein the mitochondrial uncoupler is selected from the group consisting of benzimidazoles, N-phenylanthranilates, phenylhydrazones, salicylic acids, acyldithiocarbazates, cumarines, aromatic amines that have mitochondrial uncoupling activities, and combinations thereof.

69. The therapeutic composition of claim 65, wherein the composition is incorporated into a solid or liquid dosage form.

70. A method for treating or preventing a metabolic disease or disorder in a subject, comprising administering to the subject the composition of claim 66, wherein the composition is effective for treating or preventing a metabolic disease or disorder in the subject

71. A method of treating or preventing type II diabetes or fatty liver disease in a subject, comprising administering to the subject a therapeutically effective amount of 2′,5-dichloro-4′-nitro salicylic anilide 2-aminoethanol salt, or a pharmaceutically acceptable salt thereof, wherein the symptoms or effects of type II diabetes or fatty liver disease are prevented, ameliorated or abolished.
